(19) United States
(12) Patent Application Publication
(10) Pub. No.: US 2011/0288395 A1
(43) Pub. Date: Nov. 24, 2011

(54) Pressure Measurement Device

(75) Inventors: Ahmed Elsheikh, Neston (GB); David Pye, Sydney (AU)

(73) Assignee: University of Dundee, Dundee (GB)

(21) Appl. No.: 13/125,619

(22) PCT Filed: Oct. 30, 2009
(86) PCT No.: PCT/GB2009/051464

§ 371 Date: May 23, 2011

(30) Foreign Application Priority Data
Nov. 1, 2008 (GB) 0820078.4

(51) Int. Cl. A61B 3/16

(52) U.S. Cl. 600/398

(57) ABSTRACT

A non-invasive device adapted to measure intraocular pressure (IOP) comprising: a pressure sensor mounted on a soft contact lens to be worn on the eye of a subject, wherein the device is adapted such that, when worn on the eye, the pressure sensor is located at or near the transitional region of the cornea. The pressure sensor communicates wirelessly to an external controlling device through a magnetic field-based telemetry system that serves to power the pressure sensor and transmit the pressure measurements to the external device.

The present invention relates to devices for measuring intraocular pressure.

Glaucoma is a group of diseases of the eye which, worldwide, is the leading cause of irreversible blindness. The major risk factor for the diseases is increased intraocular pressure (IOP). Therefore, testing for glaucoma includes measurements of IOP via tonometry.

It is generally accepted that the normal range of IOP is 10 to 21 mmHg, and individuals with an IOP higher than this range will usually take IOP lowering medication. Also, IOP for the individual can vary throughout a 24 hour period depending on whether the individual is asleep or awake, the level of physical exertion or hydration, or the psychological state. Therefore, measuring IOP only during irregular daytime periods, such as during two or three clinic visits a year, does not provide sufficient information for proper management of the disease.

Furthermore, all pressure measurement devices used to measure IOP are affected to different extents by the stiffness of the cornea. This stiffness varies from individual to individual due to variations in factors including thickness, curvature, age and medical history. Therefore, measured values of IOP require correction or calibration to account for these natural variations in stiffness.

A common method of measuring IOP is applanation tonometry which measures IOP by flattening a constant area of the cornea using a force applied to the cornea. Since contact is made with the cornea, an anaesthetic must be introduced onto the surface of the eye. Apart from the discomfort for the subject, clinical technicians are required to take the measurements. This also entails that measurements will be taken only during the daytime which, again, does not provide sufficient information for proper management of the disease. Typically, a small number of measurements will be taken at each visit during office hours and an average value calculated. Also, it is known that the major production of fluid which affects levels of IOP occurs during night time. Other methods of measuring IOP exist which involve non-contact tonometry, but these suffer from the same or other disadvantages.

It is desirable to provide a device for measuring IOP which substantially avoids discomfort for the subject or the need for clinical technicians and the like to perform the measuring process.

It is desirable to provide a device for measuring IOP which allows multiple measurements to be taken during a 24 hour period. It is desirable to provide a device for measuring IOP which allows substantially continuous measurements to be taken.

It is known that the cornea changes in structure as the distance from the centre of the cornea increases. In a central region, up to a diameter of approximately 6 to 8 mm, the cornea includes collagen fibrils which predominately have a vertical or horizontal orientation. In an outer region beyond this, the collagen fibrils change in orientation to a predominately circumferential orientation. It has been found that the transitional region between the central region and the outer region is characterised as being an area of low stiffness. Therefore, deformations due to changes in IOP will be most apparent at this location.

It is known to provide a semi-rigid ring or contact lens which applanates the sclera of the eye and holds a pressure sensor such as a strain gauge in contact with the sclera to measure IOP. Due to contact with, and applanation of, the naturally stiff sclera, these devices are bulky and uncomfortable to wear.

It is desirable to provide a device which measures IOP at or near the transitional region of the cornea. It is desirable to provide a device for measuring IOP which avoids applanation of the eye. It is desirable to provide a device for measuring IOP which avoids obstructing the vision of the subject.

According to a first aspect of the present invention there is provided a device adapted to measure intraocular pressure comprising:
a pressure sensor to be worn on the eye of a subject,
wherein the device is adapted such that, when worn on the eye, the pressure sensor is located at or near the transitional region of the cornea.

The pressure sensor may comprise a substantially circular member. The circular member may comprise an annular member. Alternatively, the circular member may comprise a disc member.

The pressure sensor may comprise a strain gauge having a resistance element. The strain gauge may be configured such that at least a portion of the resistance element is orientated in a radial direction relative to the centre of the cornea. At least a portion of the resistance element of the strain gauge may be orientated in a vertical direction. At least a portion of the resistance element of the strain gauge may be orientated in a horizontal direction. However, the resistance element of the strain gauge may be orientated in any direction.

The circular member may have an annular groove at a location corresponding to the transitional region of the cornea. The annular groove may be located at a distance of between 6 to 8 mm from the centre of the cornea. The strain gauge may be configured such that at least a portion of the resistance element transverses the annular groove at one or more circumferential locations of the annular groove. The circumferential location may be at a vertically radial location relative to the centre of the cornea. The circumferential location may be at a horizontally radial location relative to the centre of the cornea.

The device may include a contact lens. The contact lens may be a corneal contact lens. The contact lens may be a soft contact lens, such as formed from a hydrogel.

The circular member may be flexible, semi-rigid or rigid.

The circular member may be located at an outer surface of the contact lens. Alternatively, the annular member may be embedded within the contact lens.

The circular member may comprise an internal volume or cavity of the contact lens. The cavity may contain a liquid, such as saline.

Alternatively, the cavity may contain a medication for lowering intraocular pressure, and the circular member includes an outlet to allow medication to exit the cavity.

Valve means may be provided at the cavity to prevent or allow medication to exit the cavity. The valve means may be adapted to prevent medication to exit the cavity when the IOP is at a low or normal level, and to allow medication to exit the cavity when the IOP is at a high level.

The valve means may be adapted to allow medication to exit the cavity in response to a high level of IOP being sensed by the pressure sensor. Alternatively, the valve means may be adapted to allow medication to exit the cavity in response to deformation of the device due to a high level of IOP.

The device may include a first transceiver, such as a coil, connected to the resistance element of the strain gauge. The device may include a second remote transceiver, such as a coil, adapted to form a magnetic field with the first transceiver. The second transceiver may be connected to a power source such that power is transmitted to the first transceiver. The first transceiver may be adapted to communicate values or changes in values of the measured intraocular pressure to the second transceiver. The second transceiver may be adapted to communicate data relating to the measured intraocular pressure to a data recording device.

According to a second aspect of the present invention there is provided a device adapted to measure intraocular pressure comprising:
a corneal contact lens; and
a substantially circular member including a pressure sensor.

The contact lens may be a soft contact lens, such as formed from a hydrogel.

The circular member may be located at an outer surface of the contact lens. Alternatively, the annular member may be embedded within the contact lens.

The circular member may comprise an internal volume or cavity of the contact lens. The cavity may contain a liquid, such as saline.

Alternatively, the cavity may contain a medication for lowering intraocular pressure, and the circular member includes an outlet to allow medication to exit the cavity.

Valve means may be provided at the cavity to prevent or allow medication to exit the cavity. The valve means may be adapted to prevent medication to exit the cavity when the IOP is at a low or normal level, and to allow medication to exit the cavity when the IOP is at a high level.

The valve means may be adapted to allow medication to exit the cavity in response to a high level of IOP being sensed by the pressure sensor. Alternatively, the valve means may be adapted to allow medication to exit the cavity in response to deformation of the device due to a high level of IOP.

The circular member may comprise an annular member. Alternatively, the circular member may comprise a disc member.

The device may be adapted such that, in use, the pressure sensor is located at or near the transitional region of the cornea.

The pressure sensor may comprise a strain gauge having a resistance element. The strain gauge may be configured such that at least a portion of the resistance element is orientated in a radial direction relative to the centre of the cornea. At least a portion of the resistance element of the strain gauge may be orientated in a vertical direction. At least a portion of the resistance element of the strain gauge may be orientated in a horizontal direction.

The circular member may have an annular groove. The strain gauge may be configured such that at least a portion of the resistance element transverses the annular groove at one or more circumferential locations of the annular groove. The circumferential location of the annular groove may be at a vertically radial location relative to the centre of the cornea. The circumferential location of the annular groove may be at a horizontally radial location relative to the centre of the cornea.

The device may include a first transceiver, such as a coil, connected to the resistance element of the strain gauge. The device may include a second remote transceiver, such as a coil, adapted to form a magnetic field with the first transceiver. The second transceiver may be connected to a power source such that power is transmitted to the first transceiver. The first transceiver may be adapted to communicate values or changes in values of the measured intraocular pressure to the second transceiver. The second transceiver may be adapted to communicate data relating to the measured intraocular pressure to a data recording device.

According to a third aspect of the present invention there is provided a device to be worn in the eye of a subject, the device comprising:
a cavity adapted to contain a medication for lowering intraocular pressure; and
a cavity outlet to allow medication to exit the cavity.

The device may include a contact lens. The contact lens may be a corneal contact lens.

The device may include a pressure sensor for measuring intraocular pressure. The device may be adapted such that, when worn in the eye, the pressure sensor is located at or near the transitional region of the cornea.

The pressure sensor may comprise a substantially circular member. The circular member may comprise an annular member. Alternatively, the circular member may comprise a disc member.

The circular member may comprise the cavity.

Valve means may be provided at the cavity to prevent or allow medication to exit the cavity. The valve means may be adapted to prevent medication to exit the cavity when the IOP is at a low or normal level, and to allow medication to exit the cavity when the IOP is at a high level.

The valve means may be adapted to allow medication to exit the cavity in response to a high level of IOP being sensed by the pressure sensor. Alternatively, the valve means may be adapted to allow medication to exit the cavity in response to deformation of the device due to a high level of IOP.

The pressure sensor may comprise a strain gauge having a resistance element.

Embodiments of the present invention will now be described, by way of example only, with reference to the accompanying drawings in which:

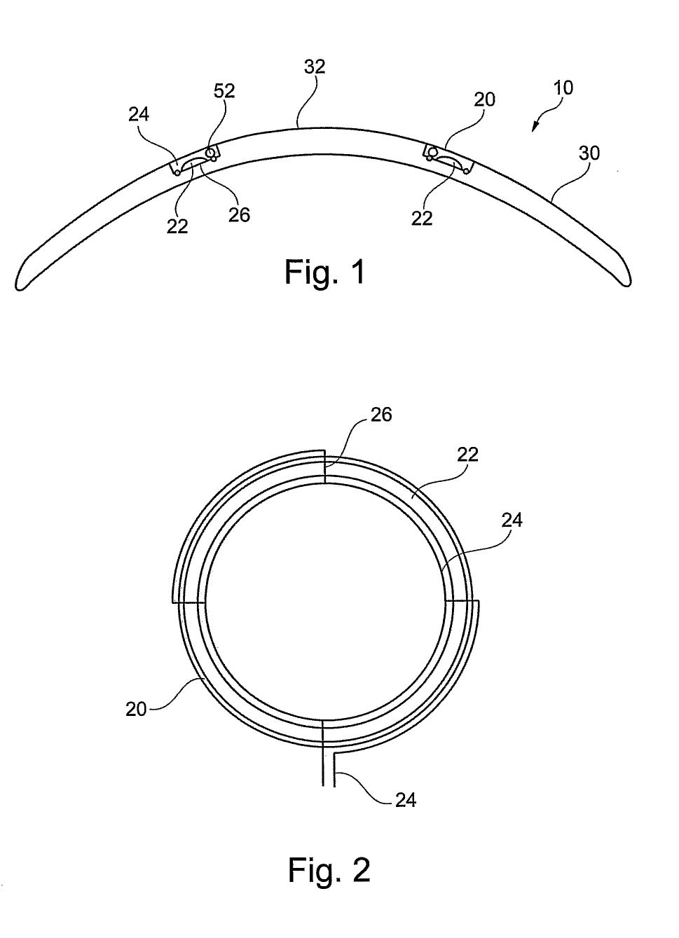

FIG. 1 is a side view of a device according to a first embodiment of the invention;

FIG. 2 is a plan view of a circular member of the device of FIG. 1;

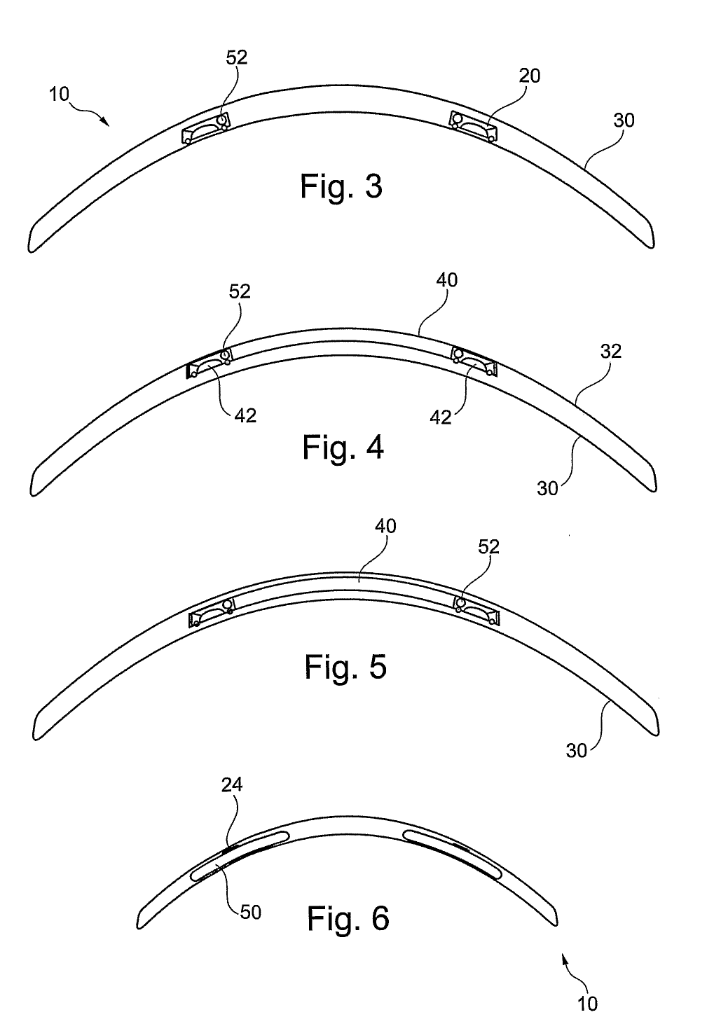

FIG. 3 is a side view of a device according to a second embodiment of the invention;

FIG. 4 is a side view of a device according to a third embodiment of the invention;

FIG. 5 is a side view of a device according to a fourth embodiment of the invention;

FIG. 6 is a side view of a device according to a fifth embodiment of the invention; and

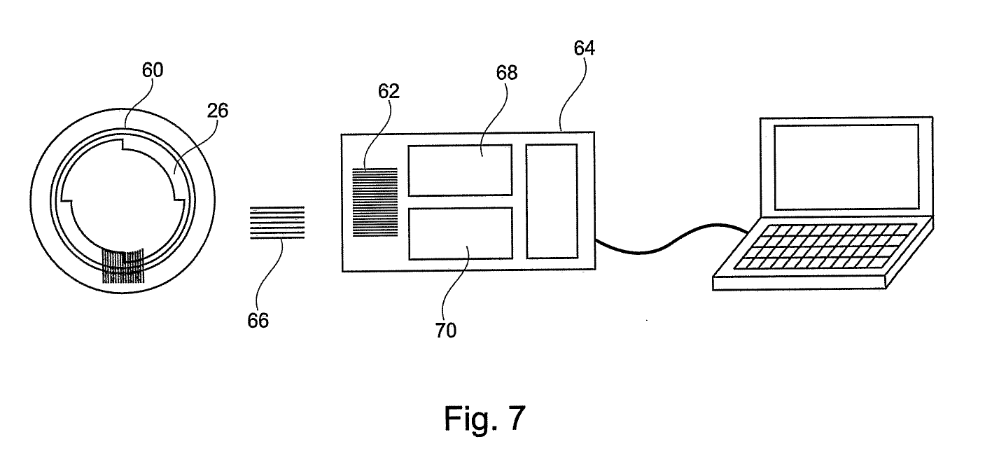

FIG. 7 is a schematic view of a device connected to an external device.

In a first embodiment of the invention, FIG. 1 shows a device 10 which is adapted to measure the intraocular pressure (IOP) of a subject. The device 10 comprises a substantially circular annular member 20 which is located at an outer surface 32 of a corneal contact lens 30. The contact lens 30 is formed from a soft material such as a hydrogel.

The annular member 20 is semi-rigid and has an annular groove 22. The annular member 20 includes a pressure sensor in the form of a strain gauge 24 which has a resistance element or wire 26.

As shown in FIG. 2, the wire 26 firstly extends around an outer diameter of the annular member 20 but then transverses the annular groove 22 at a circumferential location of the annular groove 22 which is horizontally radial relative to the centre of the annular member 20. The wire 26 then extends within an inner diameter of the annular member 20 before transversing the annular groove 22 at a circumferential location which is vertically radial relative to the centre of the annular member 20. This pattern is repeated such that the wire 26 transverses the annular groove 22 at four circumferential locations, two of which are vertically radial and two of which are horizontally radial relative to the centre of the annular member 20.

The annular member 20, and in particular the annular groove 22, has a diameter which corresponds to the transitional region of the cornea when the device is placed in the eye of the subject. Therefore, the strain gauge 24 is configured to measure IOP at a location where changes in IOP will be most apparent. Furthermore, a radial arrangement of the wire 26 of the strain gauge 24 is the most suitable for measuring the expansion or contraction of a substantially spherical body.

FIG. 3 shows a second embodiment similar to the first except that the annular member 20 is embedded within the contact lens 30. This can provide increased comfort for the subject.

FIG. 4 shows a third embodiment in which the annular member 20 has been replaced by a disc member 40. The disc member 40 is located at the outer surface 32 of a corneal contact lens 30. The disc member 40 still has an annular groove 42 at a location which corresponds to the transitional region of the cornea when the device 10 is placed in the eye of the subject.

FIG. 5 shows a fourth embodiment similar to the third except that the disc member 40 is embedded within the contact lens 30.

FIG. 6 shows a fifth embodiment. The circular member comprises an internal volume or cavity 50 of the contact lens 30. This internal volume 50 is filled with a liquid and the strain gauge 24 is located at this cavity 50. This embodiment provides improved accommodation of any variation in corneal topography and less reliance on lens centration. Also, the presence of the liquid results in a uniform pressure within the cavity 50 and, since the strain gauge 24 is located here, this uniform pressure is sensed by the strain gauge 24.

It is to be appreciated that the cavity can be provided separate from the circular member. In the embodiments of FIGS. 1 to 5, a separate cavity 52 is shown.

The cavity 50, 52 may be filled with saline. However, in an alternative embodiment, the cavity contains a medication for lowering IOP. In such case, a cavity outlet (not shown) is provided to allow the medication to exit the cavity.

A valve (not shown) can be provided at the cavity outlet to prevent or allow the medication to exit the cavity 50. When the IOP is at a low or normal level, the valve is closed to prevent medication from exiting the cavity 50. However, when the IOP is at a high level, the valve opens to allow medication to exit the cavity 50.

The valve may open in response to a high level of IOP being sensed by the pressure sensor.

Alternatively, the valve may open in response to deformation of the contact lens 30 due to a high level of IOP. For example, the valve may comprise two flaps provided at the cavity outlet which abut or overlap when the contact lens 30 is substantially non-deformed. However, the contact lens 30 may be adapted such that a high level of IOP causes the contact lens 30 to deform which causes the flaps to move apart, thereby allowing medication to exit the cavity 50. The greater the IOP, the greater the moving apart of the flaps, and thus the greater the flow of medication from the cavity 50.

In each of the embodiments described, the device 10 can include a first transceiver or coil 60 which is connected to the wire 26 of the strain gauge 24. A second remote transceiver, also a coil 62 which is part of an external device 64, is adapted to form a magnetic field 66 with the first coil 60. The external device 64 and second coil 62 can be connected to a power source 68 so that power is transmitted to the first coil 60 for operation of the strain gauge 24.

The second coil 62 will also be responsive to changes in the resistance of the first coil 60 caused by changes in the measured IOP. The response of the second coil 62 therefore corresponds to IOP data and this data can be recorded using a data recording device 70. This allows the continuous measurement of IOP.

In an embodiment in which the contact lens 30 includes a cavity 50 containing medication for lowering IOP and a valve which opens in response to a high level of IOP being sensed by the pressure sensor, the valve can be an electronic valve. The opening and closing of the electronic valve can be controlled by the external device 64 using a control signal transmitted by the second coil 62. In such an embodiment, the amount of medication released can be accurately controlled.

Whilst specific embodiments of the present invention have been described above, it will be appreciated that departures from the described embodiments may still fall within the scope of the present invention.

1. A device adapted to measure intraocular pressure comprising:
a pressure sensor to be worn on the eye of a subject,
wherein the device is adapted such that, when worn on the eye, the pressure sensor is located at or near the transitional region of the cornea.

2-4. (canceled)

5. A device as claimed in claim 1, wherein the pressure sensor comprises a strain gauge having a resistance element.

6. A device as claimed in claim 5, wherein the strain gauge is configured such that at least a portion of the resistance element is orientated in a radial direction relative to the centre of the cornea.

7. A device as claimed in claim 5, wherein at least a portion of the resistance element of the strain gauge is orientated in a vertical direction.

8. A device as claimed in claim 5, wherein at least a portion of the resistance element of the strain gauge is orientated in a horizontal direction.

9. A device as claimed in claim 5, wherein the circular member has an annular groove at a location corresponding to the transitional region of the cornea.

10. A device as claimed in claim 9, wherein the strain gauge is configured such that at least a portion of the resistance element transverses the annular groove at one or more circumferential locations of the annular groove.

11. A device as claimed in claim 1, including a contact lens.

12. A device as claimed in claim 11, wherein the contact lens is a corneal contact lens.

13. A device as claimed in claim 11, wherein the circular member is located at an outer surface of the contact lens.

14. A device as claimed in claim 11, wherein the circular member is embedded within the contact lens.

15. A device as claimed in claim 14, wherein the circular member comprises a cavity of the contact lens.

16. A device as claimed in claim 15, wherein the internal volume contains a liquid.

17. A device as claimed in claim 15, wherein the cavity contains a medication for lowering intraocular pressure, and the circular member includes an outlet to allow medication to exit the cavity.

18. A device as claimed in claim 17, wherein valve means is provided at the cavity to prevent or allow medication to exit the cavity.

19. A device as claimed in claim 18, wherein the valve means is adapted to allow medication to exit the cavity in response to a high level of IOP being sensed by the pressure sensor.

20. A device as claimed in claim 18, wherein the valve means is adapted to allow medication to exit the cavity in response to deformation of the device due to a high level of TOP.

21. A device as claimed in claim 1, including a first transceiver connected to the resistance element of the strain gauge and a second remote transceiver adapted to form a magnetic field with the first transceiver.

22. A device as claimed in claim 21, wherein the second transceiver is connectable to a power source such that power is transmitted to the first transceiver.

23. A device as claimed in claim 21, wherein the first transceiver is adapted to communicate values or changes in values of the measured intraocular pressure to the second transceiver, and wherein the second transceiver is adapted to communicate data relating to the measured intraocular pressure to a data recording device.

24-45. (canceled)
